(19) United States
(12) Patent Application Publication
(10) Pub. No.: US 2004/0161779 A1
(43) Pub. Date: Aug. 19, 2004

(54) Methods, compositions and computer software products for interrogating sequence variations in functional genomic regions

(75) Inventor: Thomas R. Gingeras, Encinitas, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/712,322

(22) Filed: Nov. 12, 2003

Related U.S. Application Data
(60) Provisional application No. 60/425,879, filed on Nov. 12, 2002.
(60) Provisional application No. 60/425,880, filed on Nov. 12, 2002.
(60) Provisional application No. 60/442,045, filed on Jan. 22, 2003.

(51) Int. Cl. C12Q 1/68

(52) U.S. Cl. 435/6

(57) ABSTRACT

In one aspect of the invention, methods, compositions and computer software products are provided to detect genetic variations in functional regions of a genome. In some embodiments, oligonucleotide tiling probe arrays are used to detect transcribed regions, transcription factor binding regions, etc. Genetic variations in these regions can be determined with, e.g., a whole genome sampling assay.

### RELATED APPLICATIONS

This application claims the priority of U.S. Provisional Application Serial Nos. 60/425,879 and 60/425,880, filed on Nov. 12, 2002, which are incorporated herein by reference for all purposes.

### FIELD OF THE INVENTION

The present invention relates to genetic analysis, genomics, biological assays and bioinformatics. Specifically, in one aspect of the invention, methods, compositions and computer software products are provided for analyzing genetic variations in functional regions.

### BACKGROUND OF THE INVENTION

The analysis of the genome for variations (e.g. single nucleotide polymorphisms or SNPs, amplifications and deletions) which may be the cause of interesting biology has focused on the combination of locating such variations in the genome of affected individuals and correlating such variations to the annotations listed in those regions (e.g. coding regions and regulatory regions). Thus, searches for genotype:phenotype correlations have depended upon the existing annotations of the genome. There is, however, also a need, for example, to monitor the unannotated portions of the genome in order to obtain an unbiased coverage of the transcriptional activity of the genome.

### SUMMARY OF THE INVENTION

In one aspect of the invention, methods, compositions and computer software products are provided for facilitating searches for sequence variations (SNPs, amplifications, deletions etc.) in functional regions of the genome, without recourse to existing annotations.

In some embodiments, RNA transcription sites, transcription factor binding sites, origins, methylation and chromatin modification sites, etc. are determined in biological samples. Typically, the samples may reflect various physiological, pathological, toxicological or pharmacological states. The RNA transcription sites, transcription factor binding sites, origins, methylation and chromatin modification sites, etc. confer specific functions on these genomic regions and ascribe to them a priority status for analyzing the presence of sequence variations. Regions such as these, associated with specific biological functions, are referred to as “functional regions” in this specification. The functional regions can be determined using a variety of methods including the use of high density oligonucleotide probe arrays. Typically, the sequence variations are determined on a large scale, for example, at least 500, 1000, 5000, 10000, or 100000 SNPs.

For example, when association or linkage studies highlight several regions of the genome as possible sites which may be involved in determining a trait in affected families or individuals, the presence of the functional regions in these regions can be empirically determined and can narrow down the possibilities for further analysis. The genomic and cDNA sequences in these regions can be empirically determined and can be analyzed by sequencing or SNP testing or Comparative Genomic Hybridization (CGH) testing in preference to other regions which will be important (but not exclusive) for sequence variations which are outside of coding regions.

Transcription factor binding sites can be detected using a variety of methods including the use of high density oligonucleotide probe arrays. In one embodiment, DNA fragments protected by transcription binding factors are obtained using immunoprecipitation and interrogated using high density arrays to determine regions with DNA sequences that are bound with transcriptional binding factors.

Once such functionally important sites along the genome are mapped in a few individuals, it would be useful not to have to conduct similar immunoprecipitation experiments for every factor or functional sequence in every patient. For example, if several TFs (e.g. cMyc and SP-1) seem to bind to the same site (i.e. a 1 kb genomic sequence) in the genome, testing this region for mutations using the Whole Genome Sampling Assay (WGSA) would be helpful. However, if there are many such common sites scattered along the genome, then finding the fewest restriction endonucleases (REs) which allows for surveying the largest possible number of these will become a priority.

WGSA is an assay that reduces the complexity of a genomic sample by obtaining representative restriction fragments. For a detailed description of the Whole Genome Sampling Assay, see e.g., U.S. patent application Ser. Nos. 10/316,517 and 10/316,629 (incorporated herein by reference). The reduced-complexity genomic samples can be used for hybridization with high density oligonucleotide probe arrays to interrogate SNPs and perform resequencing (sequence variation detection).

As the lists of sequences involved in various functional operations of the cell such as transcription factor bindings, origins, methylation and chromatin modification sites become defined, these sites should be examined for the presence of RE sites. This is so that for a specific functional class of sequences (e.g. TF binding sequences) the fewest number of REs could be identified which would allow for surveying the largest number of such sites across the genome for possible sequence variations present in these sequences.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary process of genotyping functional regions in a genome.

FIG. 2 is a schematic showing an exemplary process of determining sequence variations in functional regions of regions identified by association or linkage analysis.

### DETAILED DESCRIPTION OF THE INVENTION

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

The invention therefore relates to diverse fields impacted by the nature of molecular interaction, including chemistry, biology, medicine and diagnostics. The ability to do so would be advantageous in settings in which large amounts of information are required quickly, such as in clinical diagnostic laboratories or in large-scale undertakings such as the Human Genome Project.

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach”1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guidefor Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Array Plate or a Plate a body having a plurality of arrays in which each array is separated from the other arrays by a physical barrier resistant to the passage of liquids and forming an area or space, referred to as a well.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5×SSPE) and a temperature of from about 25° C. to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning: A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (mRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Reader or plate reader is a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Whole Genome Sampling Assay (WGSA): An assay that allows the genotyping of thousands of SNPs simultaneously in complex DNA without the use of locus-specific primers. In this technique, genomic DNA, for example, is digested with a restriction enzyme of interest and adaptors are ligated to the digested fragments. A single primer corresponding to the adaptor sequence is used to amplify fragments of a desired size, for example, 500-2000 bp. The processed target is then hybridized to nucleic acid arrays comprising SNP-containing fragments/probes. WGSA is disclosed in, for example, U.S. Provisional Application Serial Nos. 60/319,685, 60/453,930, 60/454,090 and 60/456,206, 60/470,475, U.S. patent application Ser. Nos. 09/766,212, 10/316,517, 10/316,629, 10/463,991, 10/321,741, 10/442,021 and 10/264,945, each of which is hereby incorporated by reference in its entirety for all purposes.

III. Sequence Variations in Functional Genomic Regions

In one aspect of the invention, methods are provided for facilitating searches for sequence variations (SNPs, amplifications, deletions etc.) in functional regions of the genome without recourse to annotations. Mapping RNA and transcription factor binding sites etc. in genomic regions of affected and unaffected individuals confers specific functions on these genomic regions and ascribes to them a priority status for analyzing the presence of sequence variations. The methods are particularly useful for analyzing large regions of the genome, for example, for analyzing at least 10,000 bases, 100,000 bases, 1 Mbases or 5 Mbases of the genome.

Functional regions of a genome may be determined using a variety of methods. Preferred methods include mapping with high density oligonucleotide probe arrays. Methods for mapping functional regions of a genome are described, for example, in the following U.S. patent applications and Provisional Patent Applications: 60/339,655, “Large-Scale Transcriptional Activity of the Human Genome revealed in Chromosomes 21 and 22”; Ser. No. 10/316,518, “Methods for Determining Transcriptional Activity”; 60/425,879, “Method of Interrogating for Sequence Variations in Potentially Functional Regions in the Genome”; 60/425,880, “Method of Interrogating for Sequence Variations in Potentially Functional Regions in the Genome Using Whole Genome Assay”; 60/426,868, “Dynamic Changes in the Hidden Transcriptome of the Chromosomes 21 and 22 Upon the Differentiation of the Embryonic Cancer Cell”; 60/431,356, “Methods for Deciphering Functions of a Genome”; 60/438,866, “Methods for Analyzing Global Regulation of Coding and Non-Coding RNA Transcripts Involving Low Molecular Weight RNAs”; 60/442,045, “Transcriptome Analysis”; 60/458,718, “Methods for Detecting Large Scale Antisense Transcription”; 60/469,336, “Monitoring Transcriptional Factor Binding Sites”; 60/469,207, “Human Genome Array Plates”; 60/484,849, “Methods for Analyzing Transcript Structures”; 60/486,376, “Differential Regulation of Novel Transcripts” and 60/514,314, “Identification of Novel RNAs”. All of these applications are incorporated herein by reference for all purposes.

Functional regions may be dynamic in nature. For example, transcription sites, transcription factor binding sites may change in different physiological, pathological, toxicological and pharmacological state of a sample (see, e.g., U.S. Provisional Application Ser. No. 60/486,376, “Differential Regulation of Novel Transcripts”, incorporated herein by reference). The functional regions may also be tissue specific. In some embodiments, the functional regions may be profiled in a variety of samples of interested states to determine functional region profiles for sequence variation analysis.

The information about functional regions (such as functional region profiles) may be stored in a computerized data base. Sequence variation detection assays, such as WGSA assays may be designed using such a data base.

The genomic and cDNA sequences in the functional regions can be analyzed by sequencing or SNP testing or Comparative Genomic Hybridization (CGH) testing in preference to other regions which will be important (but not exclusive) for sequence variations which are outside of coding regions. Typically, the functional regions to be analyzed for sequence variations are at least 1000 bases, 10000 bases, 100000 bases, 1 Mbases, or 5 Mbases of the genome.

Sequencing can be performed by traditional Sanger sequencing, Sequencing by Hybridization or microarray based resequencing. For example, resequencing microarrays (Affymetrix, Santa Clara, Calif.) can be used to detect sequence variations in genomic regions. For a description of high throughput resequencing technology using microarrays, see, e.g., Warrington et al., New developments in high-throughput resequencing and variation detection using high density microarrays, Hum Mutat. 2002 April; 19(4):402-9 and U.S. patent application Ser. No. 10/028,482, both incorporated herein by reference.

SNP genotyping can be performed by a variety of methods (for a review of SNP genotyping methods, see, e.g., Pui-Yan Kwok, 2001, Methods For Genotyping Single Nucleotide Polymorphisms, Annual Review of Genomics and Human Genetics 2:235-258, and Tsuchihashi and Dracopoli, 2002, Progress in high throughput SNP genotyping methods, Pharmacogenomics J. 2002;2(2): 103-10, all incorporated here by reference). One particularly preferred method is the Whole Genome Sampling Assay (WGSA) and high density oligonucleotide probe arrays. Patent specifications disclosing WGSA have been previously incorporated by reference. The method is also described in, e.g., Kennedy et al., Large-scale genotyping of complex DNA, Nat Biotechnol. 2003 October; 21(10):1233-7, incorporated herein by reference). Typically, a large number of SNPs, such as more than 1000, 10000 or 1000000 SNPs are genotyped.

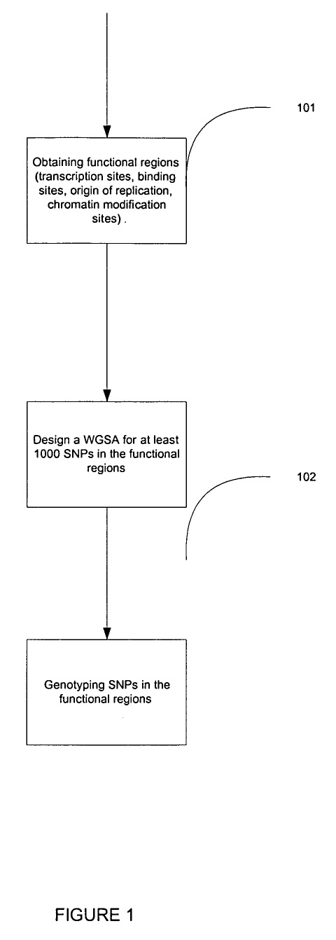

In one embodiment, computer software products are provided for designing WGSA assays. As the lists of sequences involved in various functional operations of the cell such as transcription factor bindings, origins, methylation and chromatin modification sites become defined, these sites should be examined, computationally, for the presence of restriction endonuclease (RE) sites (FIG. 1) to design a WGSA assay. This is so that for a specific functional class of sequences (e.g. transcription factor (TF) binding sequences) the fewest number of REs could be identified which would allow for surveying the largest number of such sites across the genome for possible sequence variations present in these sequences. The computer software products typically contain a computer readable medium having computer codes that perform the method of retrieving information about functional regions, analyzing RE sites suitable for interrogating the functional regions and optionally selecting probes for interrogating SNPs within the regions.

Oligonucleotides for interrogating SNPs within the functional regions are also provided. The probes are typically designed using a computer software to identify the SNPs to be interrogated. The probes are selected according to previously disclosed tiling strategies (See, e.g., Kennedy et al., Large-scale genotyping of complex DNA, Nat Biotechnol. 2003 October; 21(10):1233-7, incorporated herein by reference) or other suitable detection strategies. The probes are typically immobilized on a substrate, beads or optical fibers. In preferred embodiments, the probes are immobilized on a substrate at high densities, such as more than 1000, 100,000, 1000000 different probes per cm². Methods for manufacturing high density oligonucleotide probe arrays are described in patent specifications previously incorporated by reference.

Comparative genomic hybridization (CGH) is a molecular cytogenetic technique that allows detection of DNA sequence copy number changes throughout the genome or in specific regions of the genome in a single hybridization. For a description of CGH, see, e.g., Kallioniemi et al., Comparative genomic hybridization for molecular cytogenetic analysis of solid tumors, Science. 1992 Oct. 30; 258(5083):818-21, incorporated herein by reference).

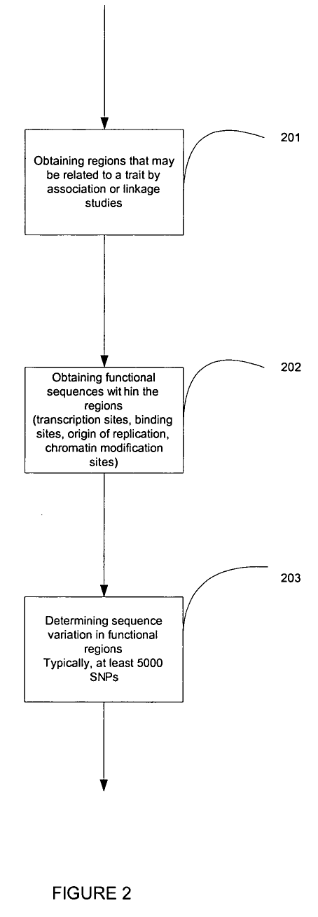

Understanding sequence variations (such as SNPs) in functional genomic regions has practical utility in, for example, drug target identification, and diagnostics. Genetic association and linkage analysis are frequently used to identify genomic regions related to a trait of interest (such as a site related to a disease). Association analysis is a method of genetic analysis that compares the frequency of alleles between affected and unaffected individuals (as used herein, an individual can be a human, an animal, a plant, etc). A given allele is considered to be associated with the disease of interest if that allele occurs at a significantly higher frequency among affected individuals. Linkage analysis is commonly used to identify the presence of a disease allele at a locus that is co-inherited with a closely linked marker, such as an SNP. Both association and linkage analysis can be performed using, e.g., WGSA, to identify a genomic region of interest. In some embodiments, when association or linkage studies highlight several regions of the genome as possible sites which may be involved in determining a trait in affected families or individuals, the presence of the functional regions in these regions can be empirically determined and can narrow down the possibilities for further analysis (FIG. 2). The genomic and cDNA sequences in these regions can be empirically determined and can be analyzed by sequencing or SNP testing or Comparative Genomic Hybridization (CGH testing in preference to other regions which will be important (but not exclusive) for sequence variations which are outside of coding regions.

Once such functionally important sites along the genome are mapped in a few individuals, it would be useful not to have to conduct similar immunoprecipitation experiments for every factor or functional sequence in every patient. For example, if several TFs (e.g. cMyc and SP-1) seem to bind to the same site (i.e. a 1 kb genomic sequence) in the genome, testing this region for mutations using the Whole Genome Sampling Assay (WGSA) would be helpful. However, if there are many such common sites scattered along the genome, then finding the fewest restriction endonucleases (REs) which allows for surveying the largest possible number of these will become a priority.

### Conclusion

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

What is claimed is:

1. A method for interrogating genetic variations comprising:
obtaining a plurality of functional regions of the genome, wherein the functional regions comprise at least 10,000 bases;
determining sequence variations of a plurality of individuals in the functional regions of the genome.

2. The method of claim 1 wherein the functional regions comprise a plurality of transcription factor binding sites.

3. The method of claim 2 wherein the functional regions comprise a plurality of RNA:protein binding domains.

4. The method of claim 3 wherein the functional regions comprise a plurality of chromatin modification sites.

5. The method of claim 4 wherein the functional regions comprise a plurality of origins of replication.

6. The method of claim 5 wherein the functional regions comprise a plurality of DNA methylation sites.

7. The method of claim 1 wherein the obtaining comprises determining functional regions using microarrays.

8. The method of claim 7 wherein the microarrays are high density oligonucleotide arrays.

9. The method of claim 8 wherein the microarrays comprise oligonucleotide probes tiling regions of the genome.

10. The method of claim 9 wherein the determining comprises determining the sequences of the functional regions of a plurality of individuals.

11. The method of claim 9 wherein the determining comprises determining the genotypes of the functional regions of a plurality of individuals.

12. The method of claim 11 wherein the genotypes are SNP genotypes.

13. The method of claim 12 wherein the determining comprises performing a WGSA with at least one restriction enzyme that is suitable for interrogating at least one functional region.

14. The method of claim 9 wherein the determining comprises determining sequence copy number changes.

15. The method of claim 1 wherein the functional regions comprise at least 100000 bases.

16. The method of claim 15 wherein the functional regions comprise at least 500000 bases.

17. A method for interrogating genetic variations comprising:
obtaining at least one interested genomic segment;
obtaining a plurality of functional regions within the interested genomic segment, wherein the functional regions comprise at least 5,000 bases;
determining sequence variations of a plurality of individuals in the functional regions of the genome.

18. The method of claim 17 wherein the interested genomic region is determined by association or linkage analysis.

19. The method of claim 18 wherein the functional regions comprise a plurality of transcription factor binding sites.

20. The method of claim 18 wherein the functional regions comprise a plurality of RNA:protein binding domains.

21. The method of claim 18 wherein the functional regions comprise a plurality of chromatin modification sites.

22. The method of claim 18 wherein the functional regions comprise a plurality of origins of replication.

23. The method of claim 18 wherein the functional regions comprise a plurality of DNA methylation sites.

24. The method of claim 17 wherein the obtaining comprises determining functional regions using microarrays.

25. The method of claim 24 wherein the microarrays are high density oligonucleotide arrays.

26. The method of claim 25 wherein the microarrays comprise oligonucleotide probes tiling regions of the genome.

27. The method of claim 24 wherein the determining comprises determining the sequences of the functional regions of a plurality of individuals.

28. The method of claim 24 wherein the determining comprises determining the genotypes of the functional regions of a plurality of individuals.

29. The method of claim 28 wherein the genotypes are SNP genotypes.

30. The method of claim 29 wherein the determining comprises performing a WGSA with at least one restriction enzyme that is suitable for interrogating at least one functional region.

31. The method of claim 24 wherein the determining comprises determining sequence copy number changes.
